(19) United States
(12) Patent Application Publication
(10) Pub. No.: US 2017/0361156 A1
(43) Pub. Date: Dec. 21, 2017

(54) TRAINING MANAGEMENT SYSTEM, TRAINING MANAGEMENT METHOD, TRAINING MANAGEMENT PROGRAM, AND TRAINING MANAGEMENT DEVICE

(71) Applicant: SEIKO EPSON CORPORATION, Tokyo (JP)

(72) Inventor: Daisuke SUGIYA, Chino-shi (JP)

(73) Assignee: SEIKO EPSON CORPORATION, Tokyo (JP)

(21) Appl. No.: 15/544,205

(22) PCT Filed: Jan. 14, 2016
(86) PCT No.: PCT/JP2016/000169

§ 371 Date: Jul. 17, 2017

(30) Foreign Application Priority Data
Feb. 16, 2015 (JP) 2015-027280

(51) Int. Cl. A63B 24/00; A63B 71/06; A61B 5/0205; A61B 5/024; A61B 5/11

(52) U.S. Cl. CPC A63B 24/0006; A63B 24/0075; A63B 71/0622; A61B 5/02055; A63B 24/0062; A63B 2220/74; A63B 2220/72; A63B 2220/62; A63B 2220/40; A63B 2220/20; A63B 2220/12; A63B 2024/0009; A61B 2560/0257; A61B 5/1112; A61B 5/024; A63B 2220/803; A63B 2220/807; A63B 2220/836; A63B 2230/06; A63B 2225/50

(57) ABSTRACT

A system that can avoid training overemphasized by a taste of a user and prevent an injury or a trouble from being caused by an unnatural body motion by comparing, between the user and other users, evaluation values of parameters based on exercise data and analyzing training. A training management system includes data detecting means for detecting exercise data related to exercise of a user, parameter calculating means for calculating, on the basis of the detected exercise data, exercise information representing information concerning the exercise carried out by the user, evaluation-value calculating means for calculating an evaluation value of the user on the basis of the exercise information, comparison-target acquiring means for acquiring evaluation values of other users, and comparing means for comparing the evaluation value of the user and the evaluation values of the other users.

### TECHNICAL FIELD

The present invention relates to a training management system, a training management method, a training management program, and a training management device.

### BACKGROUND ART

There has been a technique of a recommendation service for recommending an item based on a taste of a user using implicit behavior history information of the user.

In recent years, there has been proposed a device that recommends, using a social network service via the Internet, an item matching a taste of a user on the basis of taste information of the user referring to review comments of a larger number of reviewers concerning items as described in PTL 1 below.

In such a recommendation service, the item to be recommended is not limited to a commodity. A case can also be assumed in which a service used by the user is recommended. For example, when the user performs training in a sports gym or the like, the recommendation service can also be applied when a training menu matching a purpose of the user is selected.

### CITATION LIST

### Patent Literature

### PTL 1: JP-A-2013-171436

### SUMMARY OF INVENTION

### Technical Problem

However, when the taste of the user overemphasizes a specific training menu, contents of training are sometimes biased. When the user selects a training menu inappropriate for the user, a large load is sometimes applied to the body of the user. An injury or a trouble is likely to be caused.

### Solution to Problem

The present invention has been devised in order to solve at least a part of the problems described above and can be realized as the following forms or application examples.

### APPLICATION EXAMPLE 1

A training management system according to this application example includes: detecting means for detecting exercise data related to exercise of a subject; exercise-information calculating means for calculating, on the basis of the detected exercise data, exercise information representing information concerning the exercise carried out by the subject; evaluation-value calculating means for calculating an evaluation value of the subject on the basis of the exercise information; acquiring means for acquiring evaluation values of other subjects; comparing means for comparing the evaluation value of the subject and the evaluation values of the other subjects; and analyzing means for analyzing a comparison result of the comparison by the comparing means.

With such a configuration, the exercise data related to the exercise of the subject is detected, the exercise information is calculated on the basis of the detected exercise data, the evaluation value for the calculated exercise information is calculated, and the calculated evaluation value and the evaluation values of the other subjects are compared and analyzed. Therefore, by comparing, between the subject and the other subjects, exercise information calculated on the basis of data and analyzing training, it is possible to avoid training overemphasized by a taste of the subject and prevent an injury or a trouble from being caused by an unnatural body motion.

### APPLICATION EXAMPLE 2

In the training management system according to the application example, it is preferable that the training management system further includes proposing means for proposing a recommended training menu to the subject on the basis of an analysis result of the analysis by the analyzing means.

With such a configuration, it is possible to propose, with the proposing means, a training menu to the subject on the basis of the analysis result.

### APPLICATION EXAMPLE 3

In the training management system according to the application example, it is preferable that the proposing means proposes a plurality of training menus as the recommended training menu to be selectable by the subject.

With such a configuration, the subject can select a training menu of the subject's own will out of the plurality of training menus proposed by the proposing means. Therefore, it is possible to improve the motivation to carryout recommended training.

### APPLICATION EXAMPLE 4

In the training management system according to the application example, the exercise data may be detected by at least one of a motion sensor, a pulse sensor, a temperature sensor, an atmospheric pressure sensor, and a position sensor.

### APPLICATION EXAMPLE 5

In the training management system according to the application example, it is preferable that the exercise-information calculating means calculates exercise information set according to a training event.

With such a configuration, it is possible to calculate the exercise information corresponding to the training event.

### APPLICATION EXMPLE 6

In the training management system according to the application example, the exercise information includes a plurality of parameters, and the evaluation value weights the parameters according to the training event.

With such a configuration, it is possible to calculate the evaluation value that weights each of the parameters.

### APPLICATION EXAMPLE 7

In the training management system according to the application example, the other subjects, whose evaluation values are within a predetermined range with respect to the evaluation value of the subject, may be selected.

### APPLICATION EXAMPLE 8

In the training management system according to the application example, the subject maybe capable of setting the predetermined range.

### APPLICATION EXAMPLE 9

In the training management system according to the application example, the exercise information may include at least one of a distance, a time, exercise of arms, energy efficiency, propulsion, exercise efficiency of legs, a running distance, a running time, rotation of pedals, a tilt of a vehicle body, and a consumed calorie.

### APPLICATION EXAMPLE 10

In the training management system according to the application example, the training management system may further include display means for displaying an analysis result of the analysis by the analyzing means.

### APPLICATION EXAMPLE 11

A training management method according to this application example includes: an acquiring step for acquiring exercise data related to exercise of a subject; an exercise-information calculating step for calculating, on the basis of the detected exercise data, exercise information representing information concerning the exercise carried out by the subject; an evaluation-value calculating step for calculating an evaluation value of the subject on the basis of the exercise information; an acquiring step for acquiring evaluation values of other subjects; an comparing step for comparing the evaluation value of the subject and the evaluation values of the other subjects; and an analyzing step for analyzing a comparison result of the comparison in the comparing step.

With such a method, the exercise data related to the exercise of the subject is detected, the exercise information is calculated on the basis of the detected exercise data, the evaluation value for the calculated exercise information is calculated, and the calculated evaluation value and the evaluation values of the other subjects are compared and analyzed. Therefore, by comparing, between the subject and the other subjects, exercise information calculated on the basis of data and analyzing training, it is possible to avoid training overemphasized by a taste of the subject and prevent an injury or a trouble from being caused by an unnatural body motion.

### APPLICATION EXAMPLE 12

A training management program according to this application example for causing a computer to realize: an acquiring function for acquiring exercise data related to exercise of a subject; an exercise-information calculating function for calculating, on the basis of the detected exercise data, exercise information representing information concerning the exercise carried out by the subject; an evaluation-value calculating function for calculating an evaluation value of the subject on the basis of the exercise information; an acquiring function for acquiring evaluation values of other subjects; an comparing function for comparing the evaluation value of the subject and the evaluation values of the other subjects; and an analyzing function for analyzing a comparison result of the comparison by the comparing function.

With such a configuration, the exercise data related to the exercise of the subject is detected, the exercise information is calculated on the basis of the detected exercise data, the evaluation value for the calculated exercise information is calculated, and the calculated evaluation value and the evaluation values of the other subjects are compared and analyzed. Therefore, by comparing, between the subject and the other subjects, exercise information calculated on the basis of data and analyzing training, it is possible to avoid training overemphasized by a taste of the subject and prevent an injury or a trouble from being caused by an unnatural body motion.

### BRIEF DESCRIPTION OF THE DRAWINGS

FIG. 1 is a block diagram showing a functional configuration of a training management system according to an embodiment of the present invention.

FIG. 2 is a diagram showing a configuration example of the training management system according to the embodiment of the present invention.

FIG. 3 is a diagram showing a relation between training data and parameters.

FIG. 4 is a diagram showing a calculation example of an evaluation value in the case in which event information is running.

FIG. 5 is a diagram showing a display example of display of a comparison result.

FIG. 6 is a diagram showing a display example of display of an analysis result.

FIG. 7 is a diagram showing a determination example of a training menu.

FIG. 8 is a flowchart showing a flow of processing of a training management method.

### DESCRIPTION OF EMBODIMENTS

An embodiment of the present invention is explained below with reference to the drawings.

### Embodiment

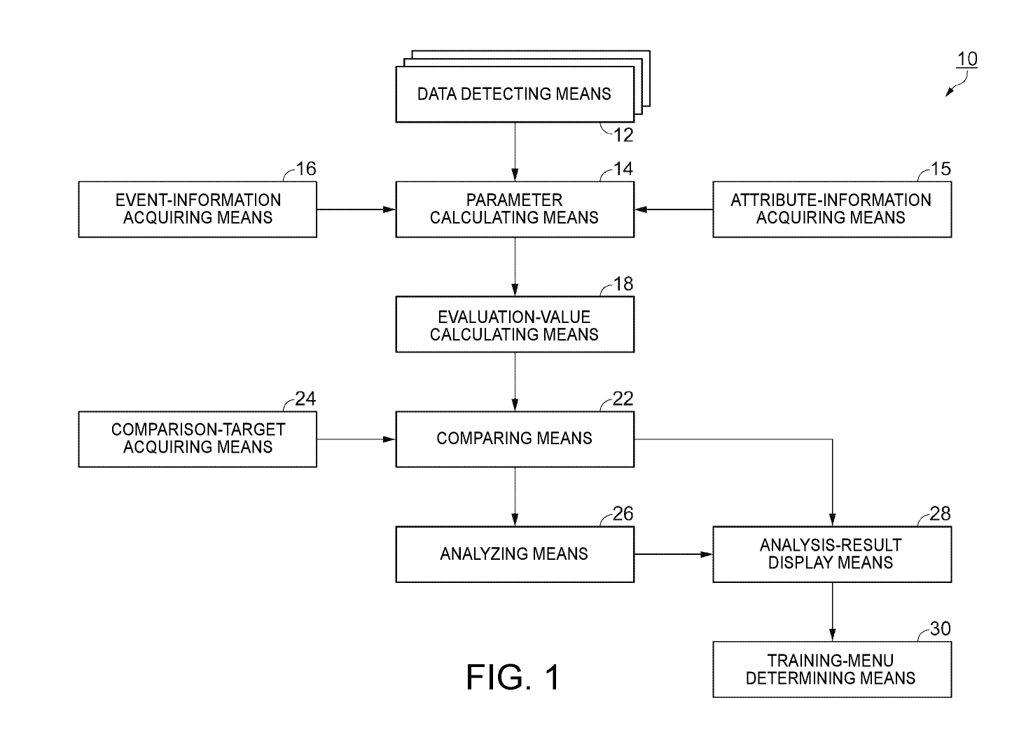

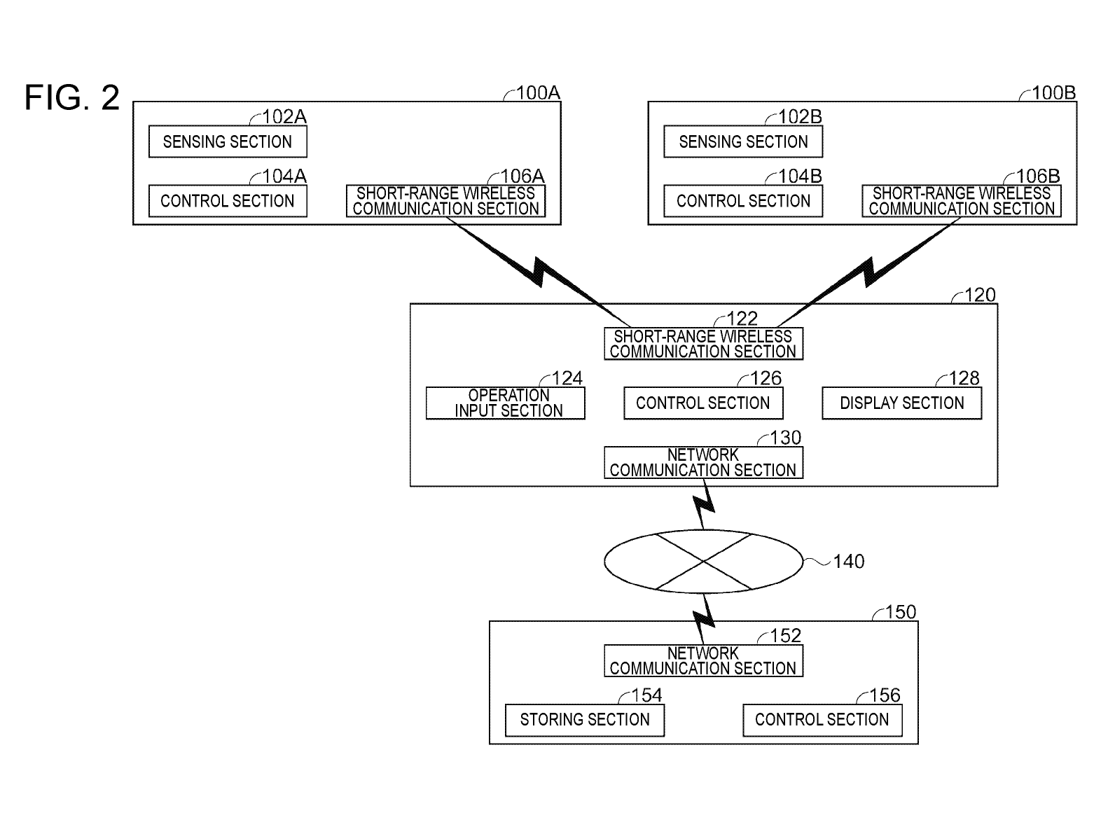

FIG. 1 is a block diagram showing a functional configuration of a training management system 10. FIG. 2 is a block diagram showing a configuration example of the training management system 10.

As shown in FIG. 1, the training management system 10 includes data detecting means 12, parameter calculating means 14, attribute-information acquiring means 15, event-information acquiring means 16, evaluation-value calculating means 18, comparing means 22, comparison-target acquiring means 24, analyzing means 26, analysis-result display means 28, and training-menu determining means 30.

As shown in FIG. 2, in this embodiment, a mode is assumed in which the training management system 10 is configured by data detecting devices 100A and 100B, a control device 120, and a server device 150.

Note that a mode for configuring the training management system 10 is not limited to the mode shown in FIG. 2. That is, the mode may be a mode in which a data detecting function and a control function are constantly configured. A mode can also be assumed in which all functions of the training management system 10 are integrally formed.

The data detecting devices 100A and 100B include sensing sections 102A and 102B, control sections 104A and 104B, and short-range wireless communication sections 106A and 106B.

The control device 120 includes a short-range wireless communication section 122, an operation input section 124, a control section 126, a display section 128, and a network communication section 130.

The server device 150 includes a network communication section 152, a storing section 154, and a control section 156.

Referring back to FIG. 1, the data detecting means 12 is attached to a body part of a user (a subject). The data detecting means 12 detects exercise data (hereinafter referred to as training data) related to a body motion obtained when the user is performing predetermined exercise. The training data detected by the data detecting means 12 is sent to the parameter calculating means 14.

Note that, in this embodiment, the data detecting means 12 is assumed to be the data detecting devices 100A and 100B. The data detecting devices 100A and 100B are respectively attached to the arms, the legs, the head, the trunk, and the like of the user.

The data detecting devices 100A and 100B shown in FIG. 2 are explained. The sensing sections 102A and 102B include at least one of motion sensors such as tilt sensors, acceleration sensors, angular velocity sensors, or gyro sensors, pulse sensors, temperature sensors, atmospheric pressure sensors, position sensors that make use of GPS satellites, and the like. The sensing sections 102A and 102B output various kinds of information detected by the sensors as training data.

Note that the sensors are desirably selected according to a training event. For example, when the training event is swimming, the gyro sensors are attached to the arms . When the training event is cycling, the gyro sensors are attached to the legs.

The control sections 104A and 104B instruct the sensing sections 102A and 102B to output the training data. The control sections 104A and 104B cause the short-range wireless communication sections 106A and 106B to transmit the training data output by the sensing sections 102A and 102B to the control device 120 by radio.

Note that the sensing sections 102A and 102B are not limited to the sensors and may be cameras or the like capable of photographing the user.

The short-range wireless communication sections 106A and 106B communicate with the short-range wireless communication section 122 of the control device 120 by radio according to a protocol of a short-range wireless communication standard such as Bluetooth (registered trademark).

Referring back to FIG. 1, the event-information acquiring means 16 acquires event information concerning events of training performed by the user. As the events, for example, running, swimming, bicycle exercise, and the like are assumed. The event information acquired by the event-information acquiring means 16 is sent to the parameter calculating means 14.

In this embodiment, a mode is assumed in which the user inputs the event information. However, a mode for estimating the event information on the basis of training data detected by the data detecting means 12 can also be assumed. Note that a method of automatically determining a behavior pattern such as event information from outputs of the sensors is disclosed in, for example, JP-A-8-240450. A mode for causing the user to select the mode information when the event information cannot be automatically determined can also be assumed.

The attribute-information acquiring means 15 acquires attribute information of the user. The attribute information is information such as age, sex, and body weight. In this embodiment, a mode is assumed in which the user inputs the attribute information. The attribute information acquired by the attribute-information acquiring means 15 is sent to the parameter calculating means 14.

The parameter calculating means 14 is equivalent to the exercise-information calculating means. The parameter calculating means 14 calculates, on the basis of the training data sent from the data detecting means 12, exercise information (parameters) corresponding to the event information sent from the event-information acquiring means 16 and the attribute information sent from the attribute-information acquiring means 15. The parameters calculated by the parameter calculating means 14 are sent to the evaluation-value calculating means 18.

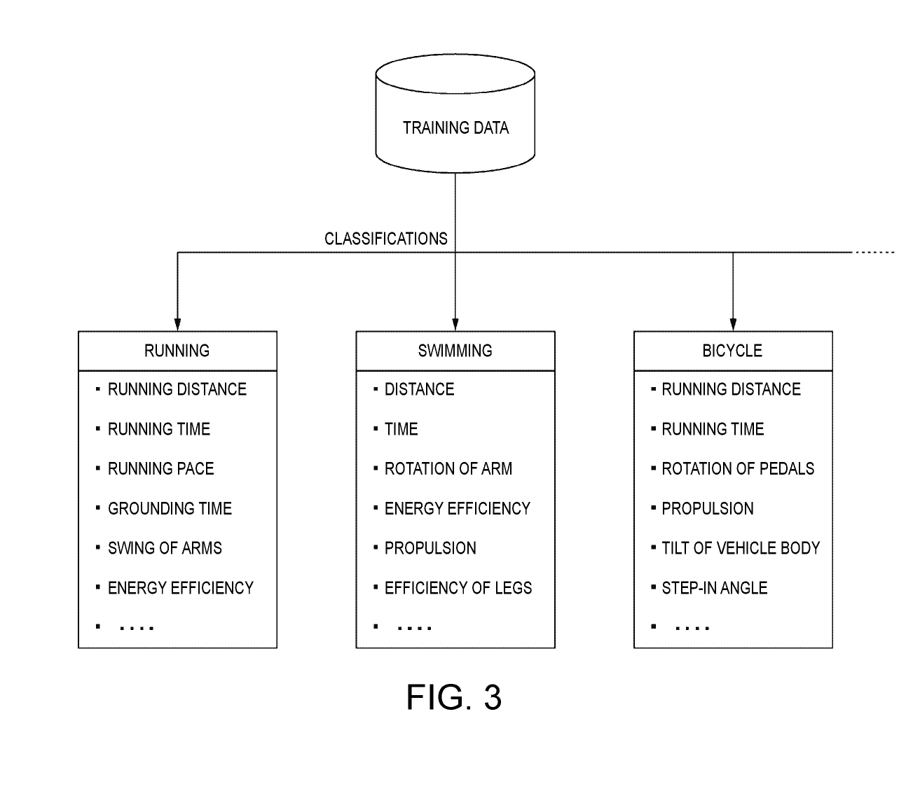

FIG. 3 shows a relation between the training data and the parameters. The parameters are specified according to a training event. For example, when the event information is the running, the parameter calculating means 14 calculates a running distance, a running time, a running pace, a grounding time, the swing of the arms, and energy efficiency from the training data as the parameters.

When the event information is the swimming, the parameter calculating means 14 calculates a distance, a time, the rotation of the arms, energy efficiency, propulsion, and exercise efficiency of the legs from the training data as the parameters.

When the event information is the bicycle exercise, the parameter calculating means 14 calculates a running distance, a running time, the rotation of pedals, propulsion, a tilt of a vehicle body, and a step-in angle from the training data as the parameters. Note that the parameters in the events include attribute information such as personal information of the user. Consequently, when a consumed calorie is set as a parameter, it is possible to calculate the consumed calorie using information concerning the body weight included in the attribute information.

Note that, as a method of calculating various parameters from output signals of the motion sensors, a well-known method can be applied. For example, as a method of calculating the parameters such as the running distance from the output signals of the motion sensors, a method disclosed in JP-A-2014-110832 can be adopted. As a method of acquiring information concerning the running from signals of the sensors, a method disclosed in JP-A-2014-6089 can be adopted. As a method of acquiring information concerning the swimming from signals of the sensors, a method disclosed in JP-A-2010-22740 can be adopted. Further, a method of acquiring information concerning the bicycle exercise from signals of the sensors, a method disclosed in JP-A-2002-272890 can be adopted.

In this embodiment, the parameter calculating means 14, the event-information acquiring means 16, and the attribute-information acquiring means 15 are assumed to be the control device 120. The control device 120 is attached to the waist of the user by, for example, a belt.

The control device 120 shown in FIG. 2 is explained. The operation input section 124 is assumed to be a touch panel or the like. The user inputs event information, attribute information, and the like to the operation input section 124.

The display section 128 displays information input to the operation input section 124 and information such as an analysis result transmitted from the server device 150.

The network communication section 130 communicates with the network communication section 152 of the server device 150 via the network 140 according to a protocol of a communication standard such as WiFi (registered trademark).

The control section 126 includes hardware such as a CPU, a RAM, and a ROM not shown in the figure and controls the operations of the sections of the control device 120. The hardware and software stored in the ROM or the like cooperate with each other, whereby the control section 126 realizes the functions of the parameter calculating means 14, the event-information acquiring means 16, and the attribute-information acquiring means 15.

Referring back to FIG. 1, the evaluation-value calculating means 18 calculates, on the basis of the parameters sent from the parameter calculating means 14, an evaluation value for training executed by the user. The evaluation value calculated by the evaluation-value calculating means 18 is sent to the comparing means 22.

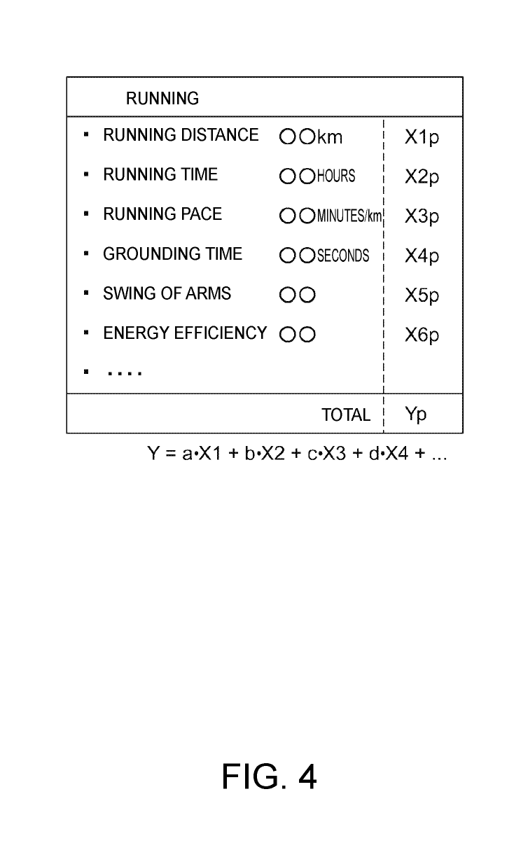

FIG. 4 shows a calculation example of an evaluation value in the case in which the event information is the running. For example, the evaluation-value calculating means 18 applies predetermined rules determined in advance and gives an X1 point (p) to the running distance. Similarly, the evaluation-value calculating means 18 gives an X2 point, an X3 point, an X4 point, an X5 point, and an X6 point respectively to the running time, the running pace, the grounding time, the swing of the arms, and the energy efficiency. When the parameters have ideal values, the numbers of points are determined on the basis of deviations from the ideal values. When a parameter has a large-is-better characteristic like the running distance, the running distance is the number of points.

Further, the evaluation-value calculating means 18 calculates a total point Y obtained by adding up the points. Note that, in this embodiment, the evaluation-value calculating means 18 calculates the total point Y taking into account weights determined in advance with respect to the parameters. For example, the evaluation-value calculating means 18 multiplies the running distance with a coefficient “a”. Similarly, the evaluation-value calculating means 18 multiplies the running time, the running pace, the grounding time, the swing of the arms, and the energy efficiency respectively with a coefficient “b”, a coefficient “c”, a coefficient “d”, a coefficient “e”, and a coefficient “f”. The evaluation-value calculating means 18 sets, as the evaluation value, the total point Y obtained by integrating multiplied values.

The comparing means 22 compares the evaluation value calculated by the evaluation-value calculating means 18 and evaluation values based on parameters of comparison target persons (other subjects) acquired by the comparison-target acquiring means 24.

Note that the comparison-target acquiring means 24 is equivalent to the acquiring means. The comparison-target acquiring means 24 may determine the comparison target persons such that differences between the evaluation values of the comparison target persons and the evaluation value of the user fit within a predetermined range. The user may determine the predetermined range. Further, the comparison-target acquiring means 24 may determine the comparison target persons on the basis of attributes of the user such as sex, age, and body weight. The comparison-target acquiring means 24 may set, on the basis of an SNS to which the user belongs, members having friendship relations with the user as the comparison target persons. A comparison result by the comparing means 22 and the evaluation value are sent to the analyzing means 26 and the analysis-result display means 28.

The analyzing means 26 analyzes training of the user on the basis of the comparison result and the evaluation value sent from the comparing means 22. A result of the analysis is sent to the analysis-result display means 28.

The analysis-result display means 28 displays the comparison result and the evaluation value sent from the comparing means 22 and the analysis result sent from the analyzing means 26.

With the training-menu determining means 30, the user determines a training menu in future referring to the displayed analysis result. In this embodiment, the training-menu determining means 30 is equivalent to the proposing means. The training-menu determining means 30 acquires information concerning training menus of a plurality of comparison target persons, whose evaluations are similar to the evaluation of the user, and proposes, on the basis of the acquired training menu, a recommended training menu estimated to be suitable for the user.

Note that the training menu determined by the user is stored in the training management system 10. The training management system 10 compares training performed by the user after the training menu determination and the determined training menu. When determining that the training deviates from the determined training menu, the training management system 10 may issue warning to the user.

Note that, in this embodiment, the evaluation-value calculating means 18, the comparing means 22, the analyzing means 26, the comparison-target acquiring means 24, and the training-menu determining means 30 are assumed to be the server device 150.

The analysis-result display means 28 is assumed to be the display section 128 and the operation input section 124 of the control device 120.

The server device 150 shown in FIG. 2 is explained.

The storing section 154 stores information concerning the parameters, the evaluation values, and the like of the comparison target persons, information concerning the comparison result, information concerning the analysis result, and information concerning the training menus. Note that the information concerning the comparison target persons includes information such as the evaluation values for the comparison target persons and the training menus performed by the comparison target persons.

The control section 156 includes hardware such as a CPU, a RAM, and a ROM not shown in the figure and controls the operations of the sections of the control device 120. The hardware and software stored in the ROM or the like cooperate with each other, whereby the control section 156 realizes the functions of the evaluation-value calculating means 18, the comparing means 22, the analyzing means 26, and the comparison-target acquiring means 24.

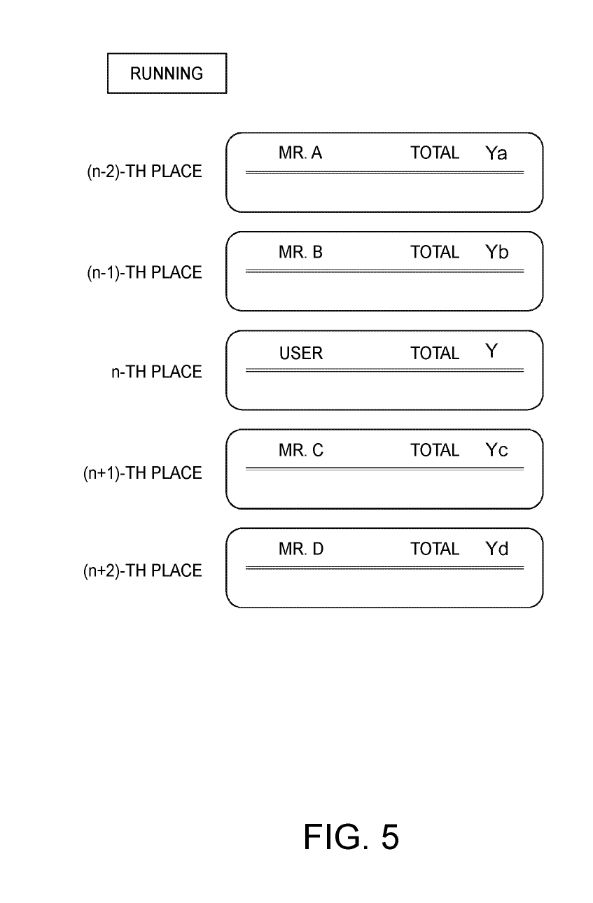

FIG. 5 shows a display example in which the analysis-result display means 28 displays the comparison result by the comparing means 22. In this case, the analysis-result display means 28 performs ranking of the comparison target persons and the user on the basis of the evaluation values and displays a rank (an n-th place) of the user and ranks of two persons before and after the user together with evaluation values of the user and the persons. Note that a range of the display can be set by the user.

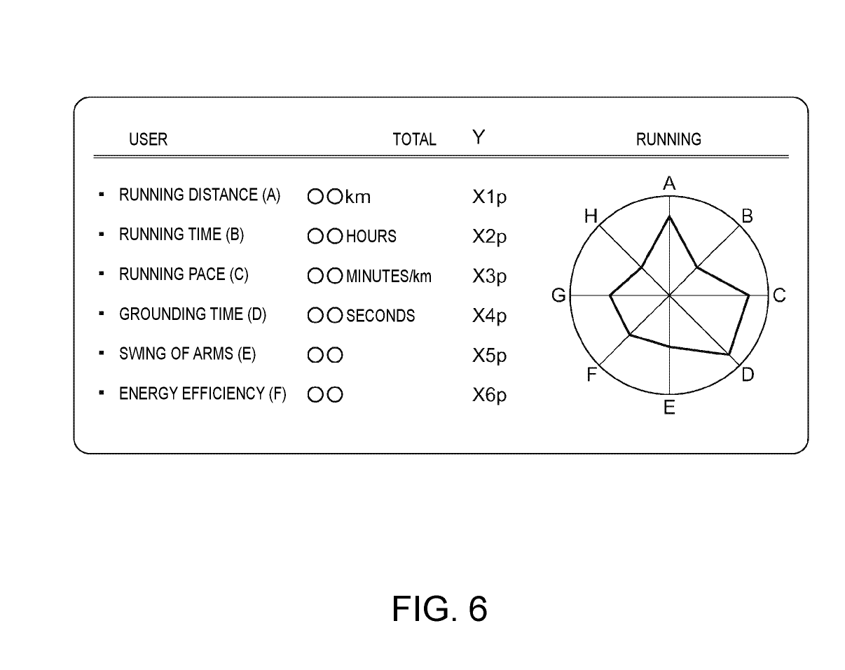

FIG. 6 shows a display example in which the analysis-result display means 28 displays the analysis result by the analyzing means 26. In this case, the analysis-result display means 28 displays evaluation values for the parameters of the user and further displays a radar chart of the parameters.

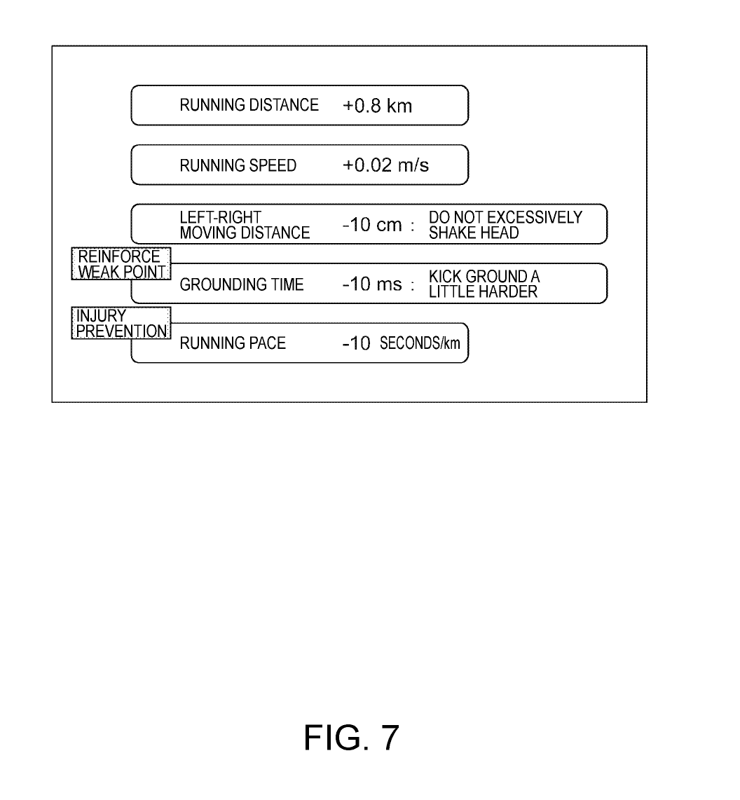

FIG. 7 shows a determination example of a training menu. The training-menu determining means 30 displays a plurality of training menus on the basis of the analysis result of the analyzing means 26 to be selectable by the user and causes the user to select a training menu. In this case, the training-menu determining means 30 may specify weak points of the user and propose a training menu recommended from the viewpoint of injury prevention based on the age and an exercise characteristic of the user in addition to a proposal of an effective training menu for improvement.

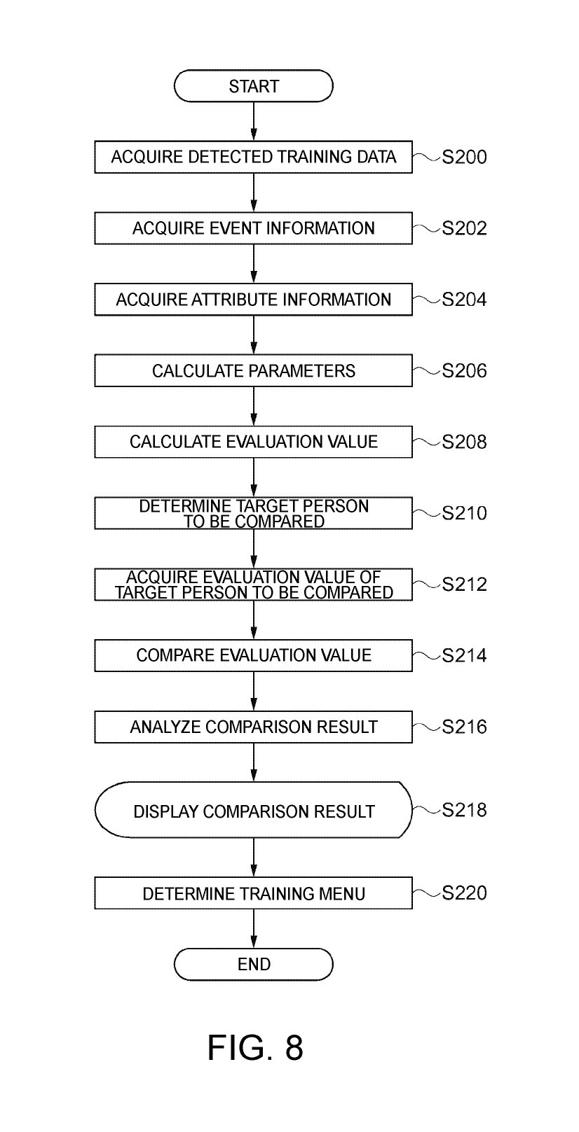

FIG. 8 is a flowchart showing a flow of processing of a training management method. When the training management system 10 is realized by a computer, the flowchart is the same as a flow of a training management program executed by the computer.

When the processing is started, the training management system 10 acquires training data detected by the sensors (step S200) <a detecting step>.

Subsequently, the training management system 10 acquires event information of training executed by the user (step S202) .

Subsequently, the training management system 10 acquires attribute information concerning the user (step S204).

Subsequently, the training management system 10 calculates parameters corresponding to the event information of the training on the basis of the training data and the attribute information (step S206) <an exercise-information calculating step>.

Subsequently, the training management system 10 calculates an evaluation value for each of the calculated parameters (step S208) <an evaluation-value calculating step>.

Subsequently, the training management system 10 determines a target person, whose evaluation value is compared with an evaluation value of the user (step S210).

Subsequently, the training management system 10 acquires the evaluation value of the target person compared with the evaluation value of the user (step S212) <an acquiring step>.

Subsequently, the training management system 10 compares the evaluation value of the user and the evaluation value of the target person (step S214) <a comparing step>.

Subsequently, the training management system 10 analyzes a comparison result (step S216) <an analyzing step> and displays an analysis result (step S218) <a display step>.

Subsequently, the training management system 10 causes the user to determine a training menu (step S220) and ends the series of processing.

According to the embodiment explained above, an effect described below is achieved.

(1) The training management system 10 detects training data according to an event of training executed by the user, calculates points for each of parameters on the basis of the detected training data, and determines a total point obtained by integrating the points. The training management system 10 can compare the total point with total points of comparison target persons to rank the user and the comparison target persons and determine a training menu to be executed by the user in future referring to training menus performed by the comparison target persons. Therefore, with the training management system 10, it is possible to determine an effective training menu for reinforcing weak points for the user. Further, it is possible to determine a training menu for preventing an injury and the like.

### REFERENCE SIGNS LIST

10 training management system

12 data detecting means

14 parameter calculating means

15 attribute-information acquiring means

16 event-information acquiring means

18 evaluation-value calculating means

22 comparing means

24 comparison-target acquiring means

26 analyzing means

28 analysis-result display means

30 training-menu determining means

100A, 100B date detecting device

102A, 102B sensing section

104A, 104B control section

106A, 106B short-range wireless communication section

120 control device

122 short-range wireless communication section

124 operation input section

126 control section

128 display section

130 network communication section

140 network

150 server device

152 network communication section

154 storing section

156 control section

1. A training management system comprising:
detecting means for detecting exercise data related to exercise of a subject;
exercise-information calculating means for calculating, on the basis of the detected exercise data, exercise information representing information concerning the exercise carried out by the subject;
evaluation-value calculating means for calculating an evaluation value of the subject on the basis of the exercise information;
acquiring means for acquiring evaluation values of other subjects;
comparing means for comparing the evaluation value of the subject and the evaluation values of the other subjects; and
analyzing means for analyzing a comparison result of the comparison by the comparing means.

2. The training management system according to claim 1, further comprising proposing means for proposing a recommended training menu to the subject on the basis of an analysis result of the analysis by the analyzing means.

3. The training management system according to claim 2, wherein the proposing means proposes a plurality of training menus as the recommended training menu to be selectable by the subject.

4. The training management system according to claim 1, wherein the exercise data is detected by at least one of a motion sensor, a pulse sensor, a temperature sensor, an atmospheric pressure sensor, and a position sensor.

5. The training management system according to claim 1, wherein the exercise-information calculating means calculates exercise information set according to a training event.

6. The training management system according to claim 5, wherein
the exercise information includes a plurality of parameters, and
the evaluation value weights the parameters according to the training event.

7. The training management system according to claim 1, wherein the other subjects, whose evaluation values are within a predetermined range with respect to the evaluation value of the subject, are selected.

8. The training management system according to claim 7, wherein the subject is capable of setting the predetermined range.

9. The training management system according to claim 1, wherein the exercise information includes at least one of a distance, a time, exercise of arms, energy efficiency, propulsion, exercise efficiency of legs, a running distance, a running time, rotation of pedals, a tilt of a vehicle body, and a consumed calorie.

10. The training management system according to claim 1, further comprising display means for displaying an analysis result of the analysis by the analyzing means.

11. A training management method comprising:
an acquiring step for acquiring exercise data related to exercise of a subject;
an exercise-information calculating step for calculating, on the basis of the detected exercise data, exercise information representing information concerning the exercise carried out by the subject;
an evaluation-value calculating step for calculating an evaluation value of the subject on the basis of the exercise information;
an acquiring step for acquiring evaluation values of other subjects;
an comparing step for comparing the evaluation value of the subject and the evaluation values of the other subjects; and
an analyzing step for analyzing a comparison result of the comparison in the comparing step.

12. A training management program for causing a computer to realize:
an acquiring function for acquiring exercise data related to exercise of a subject;
an exercise-information calculating function for calculating, on the basis of the detected exercise data, exercise information representing information concerning the exercise carried out by the subject;
an evaluation-value calculating function for calculating an evaluation value of the subject on the basis of the exercise information;
an acquiring function for acquiring evaluation values of other subjects;
an comparing function for comparing the evaluation value of the subject and the evaluation values of the other subjects; and
an analyzing function for analyzing a comparison result of the comparison by the comparing function.

13. A training management device comprising:
acquiring means for acquiring exercise data related to exercise of a subject;
exercise-information calculating means for calculating, on the basis of the exercise data, exercise information representing information concerning the exercise carried out by the subject;
evaluation-value calculating means for calculating an evaluation value of the subject on the basis of the exercise information;
acquiring means for acquiring evaluation values of other subjects;
comparing means for comparing the evaluation value of the subject and the evaluation values of the other subjects; and
analyzing means for analyzing a comparison result of the comparison by the comparing means.

14. The training management device according to claim 13, further comprising proposing means for proposing a recommended training menu to the subject on the basis of an analysis result of the analysis by the analyzing means.

15. The training management device according to claim 14, wherein the proposing means proposes a plurality of training menus as the recommended training menu to be selectable by the subject.

16. The training management device according to claim 13, wherein the exercise data is detected by at least one of a motion sensor, a pulse sensor, a temperature sensor, an atmospheric pressure sensor, and a position sensor.

17. The training management device according to claim 13, wherein the exercise-information calculating means calculates exercise information set according to a training event.
